(19) United States
(12) Patent Application Publication
(10) Pub. No.: US 2010/0175931 A1
(43) Pub. Date: Jul. 15, 2010

(54) AUTOMATIC WEIGHING DEVICE FOR HAIR DYE COLORANTS AND RELATED SYSTEM

(75) Inventor: George Amato, Mriehel (MT)

Correspondence Address: YOUNG & THOMPSON, 209 Madison Street, Suite 500, Alexandria, VA, 22314, US

(73) Assignees: INDUSTRIAL CHEMICAL COSMETICS LIMITED, MARSA (MT); BEAUTY & BUSINESS S.R.L., MILANO (IT)

(21) Appl. No.: 12/625,026

(22) Filed: Nov. 24, 2009

(30) Foreign Application Priority Data
Jan. 14, 2009 (MT) 4247

(51) Int. Cl. G01G 13/08; A61K 8/00; A61Q 5/10

(52) U.S. Cl. 177/52; 8/405

(57) ABSTRACT

A device for weighing the colorants used in the preparation of hair dyes, including a plurality of collecting bins (2) for the individual colorants, as well as an automatic system for automatically picking up the colorants from each bin, in quantities in accordance with the formulation of the dyes. Compared with the prior art methods of the field, the device of the invention offers the advantage of mechanising the operations of weighing and metering the colorants in the mixture, thus avoiding direct intervention by the operator.

The present invention concerns an automatic weighing device for the colorants used in the preparation of hair dyes. The invention further concerns the system for production of hair dyes equipped with this device.

In the production of hair dyes there is a preliminary stage of preparation of the mixture of colorants of the type and in the quantities necessary to obtain the desired hair colour.

This preparation is traditionally carried out manually, exposing the operator to contact with the colorants, usually powdery substances which are toxic on contact and by inhalation. Furthermore, manual weighing has the drawback of not ensuring precision of weighing over time. To this is added the fact that the intervention of the operator who carries out the weighing makes it impossible to maintain the secrecy of the formulation.

The main object of the present invention is to overcome the above-mentioned drawbacks of the prior art, in particular reducing manual intervention on the colorant powders, so as to essentially eliminate the risk of intoxication, also guaranteeing precision of weighing as well as ensuring the secrecy of the formulation obtained.

These and other objects are achieved with the weighing device and the system of claims 1 and 7, respectively. Preferred embodiments of the invention will be apparent from the remaining claims.

Compared with prior art methods in the field, the device of the invention offers the advantages of mechanising the operations of weighing and metering of the colorants in the mixture, thus avoiding handling of the substances by the operator and his direct intervention. A further advantage lies in the precision of the weighings, which is guaranteed over time. The device of the invention further allows preparation of the mixture of colorants to be speeded up, at the same time maintaining the secrecy of its formulation. Lastly, thanks to the system of the invention provided with the above mentioned device, it becomes possible to automate the entire hair dye production cycle.

This and other objects, advantages and characteristics are apparent from the description of a preferred embodiment of the device and the system of the present invention that follows, illustrated by way of non-limiting example in the appended figures, wherein:

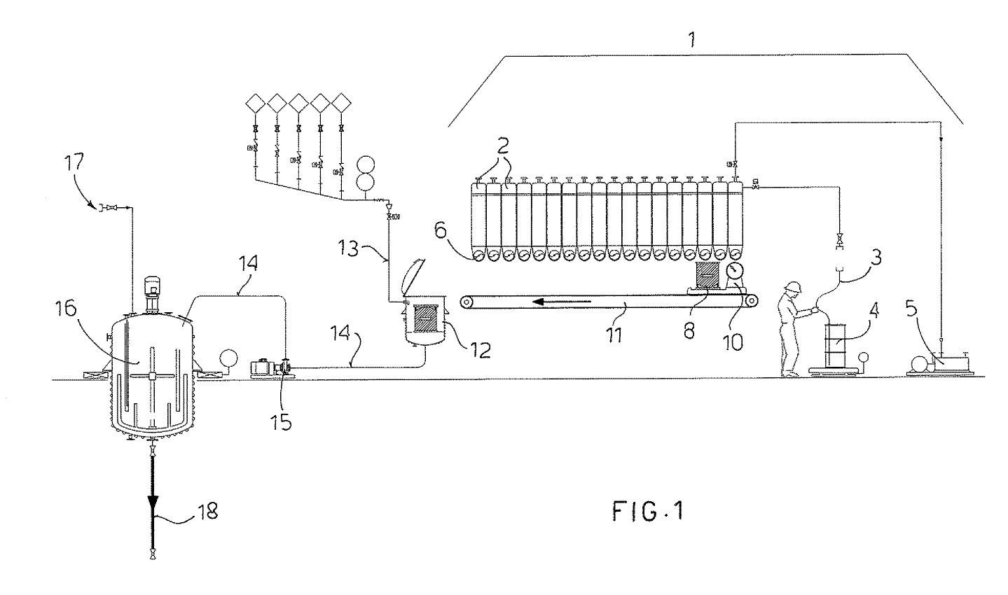

FIG. 1 shows the scheme of the system of the invention for obtaining hair dyes;

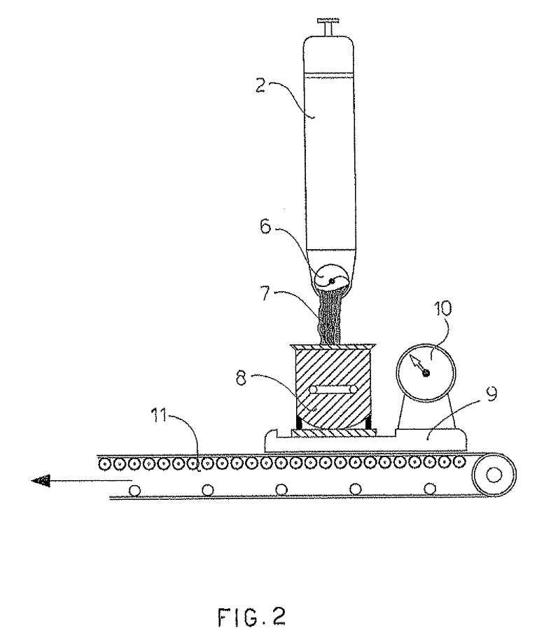

FIG. 2 shows in detail the system for automatic metering of colorants provided in the weighing device equipping the system of FIG. 1; and

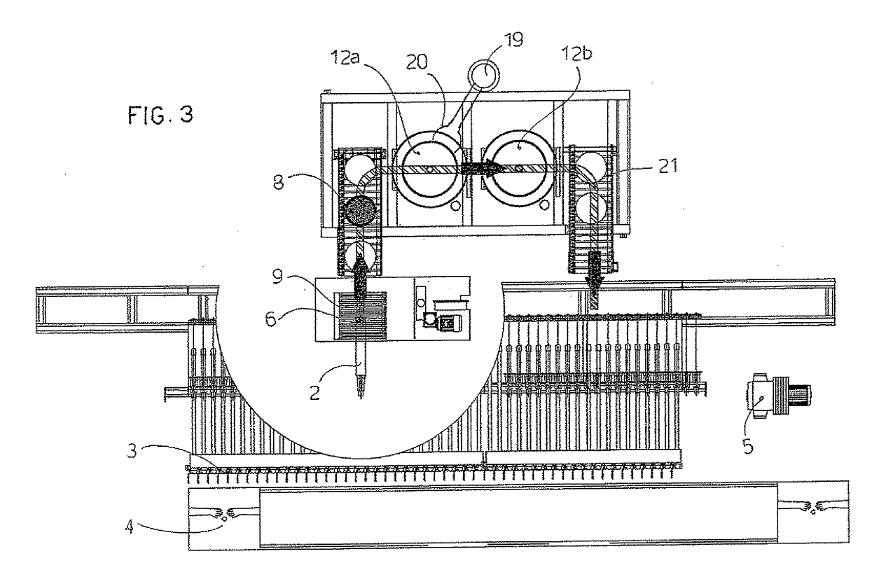

FIG. 3 shows in detail the system for transfer of the collection drum for the mixture of colorants to the subsequent stage of dissolving this mixture.

The device of the invention for weighing colorants is designated as a whole by reference numeral 1 in FIG. 1. This device comprises a plurality of bins 2, which receive the individual colorant from a suction nozzle 3, which in turn dips into a container 4 of colorant to be loaded into the respective bin. A pump 5 which maintains vacuum in the bins is also provided. Also provided on the outlet from the latter is an auger valve 6, which discharges the colorant 7 (FIG. 2) into a steel drum 8, in turn positioned on the pan 9 of a balance 10 (FIG. 2).

The balance 10 and drum 8 system is mounted moveably on a roller conveyor 11, so as to bring the same drum 8 into position beneath all the bins 2.

All the movements of opening and closing of the valves 6 of the individual bins 2, as well as those of conveying the drum 8 beneath said bins, are controlled by a computerised system (not shown), set to the formulation of the colorant mixture, in qualitative terms (which affect the shade of the colour) and in quantitative terms (which affect the final amount of dye produced).

When it leaves the weighing device 1, the drum 8 is thus loaded with the desired quantity of the mixture of colorants. At this point, the system of the invention provides for upturning of the drum 8 into a colorant mixer 12, in which the colorants are brought into contact with a flow 13 of solvents suitably metered.

Thus, at the outlet from the mixer 12, a solution of colorants 14 is obtained, which is sent to the pump 15 inside a mixer 16, also loaded with the other ingredients (fatty phase 17, etc.) suitable to obtain the final dye, in the form of a colorant cream 18.

Loading of the drum 8 into the mixer 12 is preferably carried out, as shown in FIG. 3, by means of a moveable arm or robot 19. This is provided in particular with jaws 20 for gripping the drum 8 and transferring it between a first mixer 12a for dissolving the colorant mixture, and a second mixer 12b for washing said drum 8. An automated track 21 then picks up the clean drum and positions it beneath the bins 2 again.

1. A weighing device for colorants, used in the preparation of hair dyes, characterised in that it provides means for automatic preparation of the mixture of colorants in quantities thereof in accordance with the formulation of said dyes.

2. A device according to claim 1, characterised in that said means consist in a plurality of bins (2) for collecting the individual colorants, as well as a system for automatically picking up these colorants from each bin, in the quantity necessary for preparation of said mixture.

3. A device according to claim 2, characterised in that said automatic picking up system comprises a balance (10), supported on a conveyor (11), said balance bearing a collecting drum (8) for the mixture of colorants being formed.

4. A device according to claim 3, characterised in that it further comprises valves (6) for controlling the outlet flow of colorant leaving each bin (2).

5. A device according to claim 4, characterised in that it further provides a suction nozzle system (3) and a suction pump (5) for loading the colorants into the individual bins (2).

6. A device according to claim 1, characterised in that it provides a computerised operating and control system for the valves (6) and the above-mentioned conveyor (11).

7. A system for preparation of hair dyes, characterised in that it is provided with a device according to claim 1.

8. A system according to claim 7, characterised in that it comprises a mixer (12) for preparation of the solution of said colorants, as well as a mixer (16) for preparation of the final dye.

9. A system according to claim 8, characterised in that it further comprises a moveable arm (19) for picking up the drum (8) from the conveyor (11), as well as for transferring said drum from a mixer (12a) for preparation of the colorant solution, to a mixer (12b) for washing of said drum (8).

10. A system according to claim 9, characterised in that it further provides a track (21) for transferring the empty, clean drum to said weighing device (1).

11. A process for preparing hair dyes comprising preparation of a mixture of colorants in which weighing and metering of said colorants is carried out with automatic devices, preferably with the device of claim 1.

12. A device according to claim 2, characterised in that it provides a computerised operating and control system for the valves (6) and the above-mentioned conveyor (11).

13. A device according to claim 3, characterised in that it provides a computerised operating and control system for the valves (6) and the above-mentioned conveyor (11).

14. A device according to claim 4, characterised in that it provides a computerised operating and control system for the valves (6) and the above-mentioned conveyor (11).

15. A device according to claim 5, characterised in that it provides a computerised operating and control system for the valves (6) and the above-mentioned conveyor (11).

16. A system for preparation of hair dyes, characterised in that it is provided with a device according to claim 2.

17. A system for preparation of hair dyes, characterised in that it is provided with a device according to claim 3.

18. A system for preparation of hair dyes, characterised in that it is provided with a device according to claim 4.

19. A system for preparation of hair dyes, characterised in that it is provided with a device according to claim 5.

20. A system for preparation of hair dyes, characterised in that it is provided with a device according to claim 6.
